(19) United States
(12) Patent Application Publication
(10) Pub. No.: US 2024/0173337 A1
(43) Pub. Date: May 30, 2024

(54) METHODS AND COMPOSITIONS FOR TREATING SLEEP APNEA

(71) Applicant: APNIMED, INC. (DELAWARE), Cambridge, MA (US)

(72) Inventors: Lawrence G. MILLER, Cambridge, MA (US); Luigi TARANTO-MONTEMURRO, Brookline, MA (US); Ronald FARKAS, Clarksville, MD (US)

(21) Appl. No.: 18/283,303

(22) PCT Filed: Mar. 23, 2022
(86) PCT No.: PCT/US2022/021462

§ 371 Date: Sep. 21, 2023

Related U.S. Application Data
(60) Provisional application No. 63/165,342, filed on Mar. 24, 2021.

(51) Int. Cl. A61K 31/585; A61K 31/138; A61K 31/216; A61K 45/06

(52) U.S. Cl. CPC A61K 31/585; A61K 31/138; A61K 31/216; A61K 45/06

(57) ABSTRACT

Pharmaceutical compositions comprising a norepinephtrine reuptake inhibitor (NRI) and a mineralocorticoid antagonist and optionally a muscarinic receptor antagonist (MRA) and methods of treating sleep apnea are described herein.

### CROSS-REFERENCE TO RELATED APPLICATION

This application claims priority to U.S. provisional application 63/165,342, filed Mar. 24, 2021, the entire contents of which are incorporated herein by reference.

### TECHNICAL FIELD

The present invention provides pharmaceutical compositions comprising (i) a norepinephrine reuptake inhibitor (NRI) and (ii) a mineralocorticoid antagonist, and optionally (iii) a muscarinic receptor antagonist (MRA), as well as methods of treating sleep apnea.

### BACKGROUND

Obstructive Sleep Apnea (OSA) is a common disorder caused by collapse of the pharyngeal airway during sleep. OSA can have serious health consequences.

### SUMMARY

One aspect of the present invention provides a method of treating a subject having a condition associated with pharyngeal airway collapse, the method comprising administering to a subject in need thereof an effective amount of (i) a norepinephrine reuptake inhibitor (NRI) and (ii) a mineralocorticoid antagonist.

Embodiments of this aspect of the invention may include one or more of the following optional features. In some embodiments, the NRI is a norepinephrine selective reuptake inhibitor (NSRI). In some embodiments, the NSRI is selected from the group consisting of amedalin, atomoxetine, CP-39,332, daledalin, edivoxetine, esreboxetine, lortalamine, nisoxetine, reboxetine, talopram, talsupram, tandamine, and viloxazine, or a pharmaceutically acceptable salt thereof. In some embodiments, the NRI is a norepinephrine non-selective reuptake inhibitor (NNRI) selected from the group consisting of amitriptiline, amoxapine, bupropion, ciclazindol, desipramine, desvenlafaxine, dexmethilphenidate, diethylpropion, doxepin, duloxetine, imipramine, levomilnacipran, manifaxine, maprotiline, methylphenidate, milnacipran, nefazodone, nortriptyline, phendimetrazine, phenmetrazine, protryptyline, radafaxine, tapentadol, teniloxazine, and venlafaxine, or a pharmaceutically acceptable salt thereof. In some embodiments, the NRI is selected from the group consisting of atomoxetine or a pharmaceutically acceptable salt thereof and reboxetine or a pharmaceutically acceptable salt thereof. In some embodiments, the NRI is atomoxetine or a pharmaceutically acceptable salt thereof. In some embodiments, the mineralocorticoid antagonist is selected from the group consisting of spironolactone, eplerenone, canrenone, finerenone, mexrenone, canrenoic acid, drospirenone, prorenone, apararenone, and esaxerenone, or a pharmaceutically acceptable salt thereof. In some embodiments, the mineralocorticoid antagonist is spironolactone or a pharmaceutically acceptable salt thereof. In some embodiments, the mineralocorticoid antagonist is spironolactone. In some embodiments, the method further comprises administering to the subject (iii) a muscarinic receptor antagonist (MRA). In some embodiments, the MRA is selected from the group consisting of atropine, propantheline, bethanechol, solifenacin, darifenacin, tolterodine, fesoterodine, trospium, and oxybutynin, or a pharmaceutically acceptable salt thereof. In some embodiments, the MRA is selected from the group consisting of anisotropine, benztropine, biperiden, clidinium, cycrimine, dicyclomine, diphemanil, diphenidol, ethopropazine, glycopyrrolate, hexocyclium, isopropamide, mepenzolate, methixene, methscopolamine, oxyphencyclimine, oxyphenonium, procyclidine, scopolamine, tridihexethyl, and trihexyphenidyl, or a pharmaceutically acceptable salt thereof. In some embodiments, the MRA is oxybutynin or a pharmaceutically acceptable salt thereof. In some embodiments, the MRA is (R)-oxybutynin or a pharmaceutically acceptable salt thereof. In some embodiments, the atomoxetine or pharmaceutically acceptable salt thereof is administered at a dose of from about 20 to about 200 mg. In some embodiments, the atomoxetine or pharmaceutically acceptable salt thereof is administered at a dose of from about 25 to about 100 mg. In some embodiments, the spironolactone or pharmaceutically acceptable salt thereof is administered at a dose of from about 10 to about 100 mg. In some embodiments, the spironolactone or pharmaceutically acceptable salt thereof is administered at a dose of from about 20 to about 80 mg. In some embodiments, the oxybutynin or pharmaceutically acceptable salt thereof is administered at a dose of from about 1 to about 15 mg. In some embodiments, the oxybutynin or pharmaceutically acceptable salt thereof is administered at a dose of from about 2 mg to about 10 mg. In some embodiments, the (R)-oxybutynin or pharmaceutically acceptable salt thereof is administered at a dose of from about 0.5 to about 10 mg. In some embodiments, the (R)-oxybutynin or pharmaceutically acceptable salt thereof is administered at a dose of from about 1 mg to about 5 mg. In some embodiments, the NRI and mineralocorticoid antagonist are administered in a single composition. In some embodiments, the NRI, MRA, and mineralocorticoid antagonist are administered in a single composition. In some embodiments, the single composition is an oral administration form. In some embodiments, the oral administration form is a syrup, pill, tablet, troche, capsule, or patch. In some embodiments, the condition associated with pharyngeal airway collapse is sleep apnea. In some embodiments, the condition associated with pharyngeal airway collapse is obstructive sleep apnea (OSA). In some embodiments, the condition associated with pharyngeal airway collapse is snoring. In some embodiments, the condition associated with pharyngeal airway collapse is simple snoring. In some embodiments, the subject is in a non-fully conscious state. In some embodiments, the non-fully conscious state is sleep. In some embodiments, the subject has hypertension.

Another aspect of the invention provides a pharmaceutical composition comprising (i) a norepinephrine reuptake inhibitor (NRI) and (ii) a mineralocorticoid antagonist, in a pharmaceutically acceptable carrier.

Embodiments of this aspect of the invention may include one or more of the following optional features. In some embodiments, the NRI is a norepinephrine selective reuptake inhibitor (NSRI). In some embodiments, the NSRI is selected from the group consisting of amedalin, atomoxetine, CP-39,332, daledalin, edivoxetine, esreboxetine, lortalamine, nisoxetine, reboxetine, talopram, talsupram, tandamine, and viloxazine, or a pharmaceutically acceptable salt thereof. In some embodiments, the NRI is a norepinephrine non-selective reuptake inhibitor (NNRI) selected from the group consisting of amitriptiline, amoxapine, bupropion, ciclazindol, desipramine, desvenlafaxine, dexmethilphenidate, diethylpropion, doxepin, duloxetine, imipramine, levomilnacipran, manifaxine, maprotiline, methylphenidate, milnacipran, nefazodone, nortriptyline, phendimetrazine, phenmetrazine, protryptyline, radafaxine, tapentadol, teniloxazine, and venlafaxine, or a pharmaceutically acceptable salt thereof. In some embodiments, the NRI is selected from the group consisting of atomoxetine or a pharmaceutically acceptable salt thereof and reboxetine or a pharmaceutically acceptable salt thereof. In some embodiments, the NRI is atomoxetine or a pharmaceutically acceptable salt thereof. In some embodiments, the mineralocorticoid antagonist is selected from the group consisting of spironolactone, eplerenone, canrenone, finerenone, mexrenone, canrenoic acid, drospirenone, prorenone, apararenone, and esaxerenone, or a pharmaceutically acceptable salt thereof. In some embodiments, the mineralocorticoid antagonist is spironolactone or a pharmaceutically acceptable salt thereof. In some embodiments, the mineralocorticoid antagonist is spironolactone. In some embodiments, the composition further comprises (iii) a muscarinic receptor antagonist (MRA). In some embodiments, the MRA is selected from the group consisting of atropine, propantheline, bethanechol, solifenacin, darifenacin, tolterodine, fesoterodine, trospium, and oxybutynin, or a pharmaceutically acceptable salt thereof. In some embodiments, the MRA is selected from the group consisting of anisotropine, benztropine, biperiden, clidinium, cycrimine, dicyclomine, diphemanil, diphenidol, ethopropazine, glycopyrrolate, hexocyclium, isopropamide, mepenzolate, methixene, methscopolamine, oxyphencyclimine, oxyphenonium, procyclidine, scopolamine, tridihexethyl, and trihexyphenidyl, or a pharmaceutically acceptable salt thereof. In some embodiments, the MRA is oxybutynin or a pharmaceutically acceptable salt thereof. In some embodiments, the MRA is (R)-oxybutynin or a pharmaceutically acceptable salt thereof. In some embodiments, the atomoxetine or pharmaceutically acceptable salt thereof is present in an amount of from about 20 to about 200 mg. In some embodiments, the atomoxetine or pharmaceutically acceptable salt thereof is present in an amount of from about 25 to about 100 mg. In some embodiments, the spironolactone or pharmaceutically acceptable salt thereof is present in an amount of from about 10 to about 100 mg. In some embodiments, the spironolactone or pharmaceutically acceptable salt thereof is present in an amount of from about 20 to about 80 mg. In some embodiments, the oxybutynin or pharmaceutically acceptable salt thereof is present in an amount of from about 1 to about 15 mg. In some embodiments, the oxybutynin or pharmaceutically acceptable salt thereof is present in an amount of from about 2 mg to about 10 mg. In some embodiments, the (R)-oxybutynin or pharmaceutically acceptable salt thereof is present in an amount of from about 0.5 to about 10 mg. In some embodiments, the (R)-oxybutynin or pharmaceutically acceptable salt thereof is present in an amount of from about 1 mg to about 5 mg. In some embodiments, the NRI and mineralocorticoid antagonist are formulated in a single composition. In some embodiments, the NRI, MRA, and mineralocorticoid antagonist are formulated in a single composition. In some embodiments, the single composition is an oral administration form. In some embodiments, the oral administration form is a syrup, pill, tablet, troche, capsule, or patch. In some embodiments, the composition is for use in treating a subject having a condition associated with pharyngeal airway collapse. In some embodiments, the condition associated with pharyngeal airway collapse is sleep apnea. In some embodiments, the condition associated with pharyngeal airway collapse is obstructive sleep apnea (OSA). In some embodiments, the condition associated with pharyngeal airway collapse is snoring. In some embodiments, the condition associated with pharyngeal airway collapse is simple snoring. In some embodiments, the subject is in a non-fully conscious state. In some embodiments, the non-fully conscious state is sleep. In some embodiments, the subject has hypertension.

Another aspect of the invention provides a kit comprising (i) a norepinephrine reuptake inhibitor (NRI) and (ii) a mineralocorticoid antagonist, and optionally (iii) a muscarinic receptor antagonist. In some embodiments, the kit is for use in treating a subject having a condition associated with pharyngeal airway collapse.

Another aspect of the invention provides a norepinephrine reuptake inhibitor (NRI) and a mineralocorticoid antagonist, and optionally a muscarinic receptor antagonist, for use in treating a subject having a condition associated with pharyngeal airway collapse.

Another aspect of the invention provides a therapeutic combination of (i) a norepinephrine reuptake inhibitor (NRI) and (ii) a mineralocorticoid antagonist, and optionally (iii) a muscarinic receptor antagonist, for use in treating a subject having a condition associated with pharyngeal airway collapse.

Unless otherwise defined, all technical and scientific terms used herein have the same meaning as commonly understood by one of ordinary skill in the art to which this invention belongs. Methods and materials are described herein for use in the present invention; other suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### BRIEF DESCRIPTION OF THE DRAWINGS

The following figures are provided by way of example and are not intended to limit the scope of the claimed invention.

FIG. 1 is a graphic illustration of an obstructive apnea. The top channel shows the electroencephalogram (EEG) pattern of sleep. The next channel represents airflow. The next three channels show ventilator effort by movements of the rib cage and abdomen and changes in esophageal pressure, all of which reflect a respiratory effort against an occluded upper airway. The last channel indicates oxyhemoglobin saturation.

FIG. 2 is a diagram showing the study design of Example 1.

### DETAILED DESCRIPTION

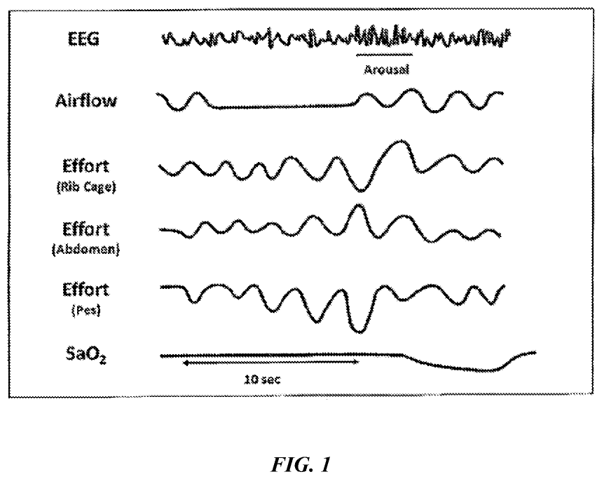

In humans, the pharyngeal airway region has no bone or cartilage support, and it is held open by muscles. When these muscles relax during sleep, the pharynx can collapse resulting in cessation of airflow. As shown in FIG. 1, ventilatory effort continues and increases in an attempt to overcome the obstruction, shown by an increase in esophageal pressure change. Rib cage and abdominal movements are in the opposite direction as a result of the diaphragm contracting against an occluded airway, forcing the abdominal wall to distend out and the chest wall to cave inward.

Increasing efforts to breathe lead to an arousal from sleep, visualisable on an EEG (FIG. 1), and result in opening of the airway and a resumption of normal breathing. The lack of airflow during the apnea also causes hypoxia, shown by a drop in oxyhemoglobin saturation (FIG. 1). Severity is generally measured using the apnea-hypopnea index (AHI), which is the combined average number of apneas (cessation of breathing for at least ten seconds) and hypopneas (reduced airflow and oxygen saturation) that occur per hour of sleep (Ruchland et al., The new AASM criteria for scoring hypopneas: Impact on the apnea hypopnea index. SLEEP 2009; 32(2):150-157).

FIG. 1 is a graphic illustration of an obstructive apnea. The top channel shows the electroencephalogram (EEG) pattern of sleep. The next channel represents airflow. The next three channels show ventilatory effort by movements of the rib cage and abdomen and changes in esophageal pressure, all of which reflect a respiratory effort against an occluded upper airway. The last channel indicates oxyhemoglobin saturation.

When a stringent definition of OSA is used (an AHI of >15 events per hour or AHI>5 events per hour with daytime sleepiness), the estimated prevalence is approximately 15 percent in males and 5 percent in females. An estimated 30 million individuals in the United States have OSA, of which approximately 6 million have been diagnosed. The prevalence of OSA in the United States appears to be increasing due to aging and increasing rates of obesity. OSA is associated with major comorbidities and economic costs, including: hypertension, diabetes, cardiovascular disease, motor vehicle accidents, workplace accidents, and fatigue/lost productivity. (Young et al., WMJ 2009; 108:246; Peppard et al., Am J Epidemiol 2013; 177:1006.)

The present leading treatment is continuous positive airway pressure (CPAP). CPAP is effective in virtually all patients, and approximately 85% of diagnosed patients are prescribed CPAP, but compliance is low. Patients find CPAP uncomfortable and often intolerable; at least 30% of patients (up to 80%) are regularly non-adherent and thus untreated (Weaver, Proc Am Thorac Soc. 2008 Feb. 15; 5(2): 173-178). Other treatment modalities with variable rates of success include oral appliances (10%) and surgery (5%), but neither is likely to be effective across the general population.

The search for medicines to activate pharyngeal muscles in sleeping humans has been discouraging; agents such as serotonin reuptake inhibitors, tricyclic antidepressants, and sedatives have all been tested in humans and shown to be ineffective at reducing OSA severity. Sec, e.g., Proia and Hudgel, Chest. 1991 August; 100(2):416-21; Brownell et al., N Engl J Med 1982, 307:1037-1042; Sangal et al., Sleep Med. 2008 July; 9(5):506-10. Epub 2007 September 27; Marshall et al. p. 2008 June; 31(6):824-31; Eckert et al., Clin Sci (Lond). 2011 June; 120(12); 505-14; Taranto-Montemurro et al., Sleep. 2017 Feb. 1; 40(2).

In a recent study, a combination of atomoxetine and oxybutynin, referred to as “ato-oxy,” administered before bedtime has been shown to reduce OSA in patients with a wide range of severity. The ato-oxy combination, which was administered for one night, reduced the number of obstructive events, improved the overnight oxygen desaturation, and enhanced the genioglossus muscle activity in a group of unselected patients with OSA. The data collected in the proof-of-concept trial showed that it was possible to improve or abolish OSA using drugs with specific neurotransmitter profiles administered systemically. See Taranto-Montemurro, Luigi et al. “The Combination of Atomoxetine and Oxybutynin Greatly Reduces Obstructive Sleep Apnea Severity. A Randomized, Placebo-controlled, Double-Blind Crossover Trial.” American journal of respiratory and critical care medicine vol. 199,10 (2019): 1267-1276.

There remains a need for further therapies for treating conditions associated with pharyngeal airway collapse such as sleep apnea.

### Methods of Treatment

The methods described herein include methods for the treatment of disorders associated with pharyngeal airway muscle collapse during sleep. In some embodiments, the disorder is sleep apnea (e.g., obstructive sleep apnea (OSA)) or snoring (e.g., simple snoring). Generally, the methods include administering a therapeutically effective amount of a norepinephrine reuptake inhibitor (NRI) and a mineralocorticoid antagonist, and optionally a muscarinic receptor antagonist (MRA), as known in the art and/or described herein, to a subject who is in need of, or who has been determined to be in need of, such treatment. In certain embodiments, the methods include administering a therapeutically effective amount of atomoxetine or a pharmaceutically acceptable salt thereof and spironolactone or a pharmaceutically acceptable salt thereof, and optionally oxybutynin (e.g., (R)-oxybutynin) or a pharmaceutically acceptable salt thereof, to a subject who is in need of, or who has been determined to be in need of, such treatment.

As used in this context, to “treat” means to ameliorate at least one symptom of the disorder associated with pharyngeal airway collapse. Often, pharyngeal airway collapse during sleep results in snoring and/or an interruption in breathing (apnea or hypopnea), arousal from sleep, and reduced oxygenation (hypoxemia); thus, a treatment can result in a reduction in snoring, apneas/hypopneas, sleep fragmentation, and hypoxemia. Administration of a therapeutically effective amount of a compound described herein for the treatment of a subject with OSA may result in decreased AHI. Measurement of OSA disease and symptoms may be, for example, by polysomnography (PSG).

In general, an “effective amount” of a compound refers to an amount sufficient to elicit the desired biological response, e.g., to treat a condition associated with pharyngeal airway collapse, e.g., to treat sleep apnea or snoring. As will be appreciated by those of ordinary skill in this art, the effective amount of a compound of the invention may vary depending on such factors as the desired biological endpoint, the pharmacokinetics of the compound, the disease being treated, the mode of administration, and the age, weight, health, and condition of the subject. An effective amount encompasses therapeutic and prophylactic treatment.

An effective amount can be administered in one or more administrations, applications or dosages. The compositions can be administered from one or more times per day to one or more times per week; including once every other day. In some embodiments, the compositions are administered daily. In some embodiments, the compositions are administered daily before sleep time, e.g., immediately before sleep time or 15-60 minutes before sleep time. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the therapeutic compounds described herein can include a single treatment or a series of treatments.

As used herein, and unless otherwise specified, a “therapeutically effective amount” of a compound is an amount sufficient to provide a therapeutic benefit in the treatment of a disease, disorder or condition, or to delay or minimize one or more symptoms associated with the disease, disorder or condition. A therapeutically effective amount of a compound means an amount of therapeutic agent, alone or in combination with other therapies, which provides a therapeutic benefit in the treatment of the disease, disorder or condition. The term “therapeutically effective amount” can encompass an amount that improves overall therapy, reduces or avoids symptoms or causes of disease or condition, or enhances the therapeutic efficacy of another therapeutic agent.

As used herein, the terms “subject” and “patient” are used interchangeably. The terms “subject” and “patient” refer to an animal (e.g., a bird such as a chicken, quail or turkey, or a mammal), specifically a “mammal” including a non-primate (e.g., a cow, pig, horse, sheep, rabbit, guinea pig, rat, cat, dog, and mouse) and a primate (e.g., a monkey, chimpanzee and a human), and more specifically a human. In one embodiment, the subject is a non-human animal such as a farm animal (e.g., a horse, cow, pig or sheep), or a pet (e.g., a dog, cat, guinea pig or rabbit). In a preferred embodiment, the subject is a human.

As used herein, “pharmaceutically acceptable” means approved or approvable by a regulatory agency of the Federal or a state government or the corresponding agency in countries other than the United States, or that is listed in the U.S. Pharmacopoeia or other generally recognized pharmacopoeia for use in animals, and more particularly, in humans.

As used herein, the term “unit dosage form” is defined to refer to the form in which the compound is administered to a subject. Specifically, the unit dosage form can be, for example, a pill, capsule, or tablet. In some embodiments, the unit dosage form is a capsule.

As used herein, “solid dosage form” means a pharmaceutical dose(s) in solid form, e.g. tablets, capsules, granules, powders, sachets, reconstitutable powders, dry powder inhalers and chewables.

For the compounds disclosed herein, single stereochemical isomers, as well as enantiomers, diastereomers, cis/trans conformation isomers, and rotational isomers, and racemic and non-racemic mixtures thereof, are within the scope of the invention. Unless otherwise indicated, all tautomeric forms of the compounds disclosed herein are within the scope of the invention.

Atomoxetine is the generic name of the pharmaceutical substance with the chemical name (-)-N-Methyl-3-phenyl-3-(o-tolyloxy)-propylamine, and its pharmaceutical salts. Atomoxetine is the R(-) isomer as determined by x-ray diffraction. In some embodiments, atomoxetine may be atomoxetine hydrochloride.

Spironolactone is the generic name of the pharmaceutical substance with the chemical name 7a-Acetylthio-17α-hydroxy-3-oxopregn-4-ene-21-carboxylic acid γ-lactone or S-[(7R,8R,9S,10R,13S,14S,17R)-10,13-dimethyl-3,5′-dioxospiro[2,6,7,8,9,11,12,14,15,16-decahydro-1H-cyclopenta[a]phenanthrene-17,2′-oxolane]-7-yl]ethanethioate, and its pharmaceutical salts.

Oxybutynin is the generic name for the pharmaceutical substance with the chemical name 4-diethylamino-2-butynylphenylcyclohexylglycolate or 4-(diethylamino)but-2-ynyl 2-cyclohexyl-2-hydroxy-2-phenylacetate, and its pharmaceutically acceptable salts. In various embodiments, oxybutynin may be a racemic mixture of R- and S- enantiomers, or an isolated enantiomer, e.g., the R-enantiomer. In various embodiments, oxybutynin may be oxybutynin chloride or (R)-oxybutynin chloride.

In some embodiments, the methods include administering a dose of from about 20 mg to about 200 mg of atomoxetine or a pharmaceutically acceptable salt thereof (or a dose equivalent of another NRI). In some embodiments, the dose of atomoxetine or a pharmaceutically acceptable salt thereof is from about 25 mg to about 100 mg. In some embodiments, the dose of atomoxetine or pharmaceutically acceptable salt thereof is from about 40 mg to about 80 mg. In some embodiments, the dose of atomoxetine or pharmaceutically acceptable salt thereof is from about 20 mg to about 50 mg. In some embodiments, the dose of atomoxetine or a pharmaceutically acceptable salt thereof is from about 50 mg to about 100 mg. In some embodiments, the dose of atomoxetine or pharmaceutically acceptable salt thereof is about 40 mg. In some embodiments, the dose of atomoxetine or pharmaceutically acceptable salt thereof is about 80 mg.

In some embodiments, the methods include administering a dose of from about 10 mg to about 200 mg of spironolactone or pharmaceutically acceptable salt thereof (or a dose equivalent of another mineralocorticoid antagonist). In some embodiments, the dose of spironolactone or a pharmaceutically acceptable salt thereof is from about 15 to about 100 mg. In some embodiments, the dose of spironolactone or a pharmaceutically acceptable salt thereof is from about 20 to about 80 mg. In some embodiments, the dose of spironolactone or a pharmaceutically acceptable salt thereof is from about 20 to about 40 mg. In some embodiments, the dose of spironolactone or a pharmaceutically acceptable salt thereof is from about 40 to about 80 mg. In some embodiments, the dose of spironolactone or a pharmaceutically acceptable salt thereof is about 25 mg. In some embodiments, the dose of spironolactone or a pharmaceutically acceptable salt thereof is about 50 mg.

In methods comprising administration of oxybutynin or (R)-oxybutynin or a pharmaceutically acceptable salt thereof (or another MRA), the dose of oxybutynin or (R)-oxybutynin or pharmaceutically acceptable salt thereof may be from about 1 mg to about 25 mg (or a dose equivalent thereof of another MRA), or in some embodiments, from about 2 mg to about 15 mg. In some embodiments, the dose of oxybutynin or pharmaceutically acceptable salt thereof is from about 2.5 mg to about 10 mg, e.g., 5 mg. In some embodiments, the dose of (R)-oxybutynin or pharmaceutically acceptable salt thereof is from about 1 mg to about 5 mg, e.g., 2.5 mg. In some embodiments, the dose of oxybutynin or (R)-oxybutynin or pharmaceutically acceptable salt thereof is from about 1 mg to about 10 mg.

In some embodiments, the methods include administering 40 mg atomoxetine hydrochloride and 25 mg spironolactone. In some embodiments, the methods include administering 40 mg atomoxetine hydrochloride and 50 mg spironolactone. In some embodiments, the methods include administering 80 mg atomoxetine hydrochloride and 25 mg spironolactone. In some embodiments, the methods include administering 80 mg atomoxetine hydrochloride and 50 mg spironolactone.

In some embodiments, the methods further comprise administering (iv) an additional active agent, which is a diuretic. The (iv) diuretic may be used with or without an MRA. In some embodiments, the diuretic is selected from the group consisting of chlorothiazide, hydrochlorothiazide, bendroflumethiazide, chlorthalidone, methychlothiazide, polythiazide, triamterene, furosemide, ethacrynic acid, metolazone, bumetanide, indapamide, amiloride, and torsemide, or a pharmaceutically acceptable salt thereof.

In some embodiments, the subject has hypertension. In some embodiments, the subject is in a fluid-retaining state. For example, the fluid retaining-state may be due to hypertension, heart failure, or end-stage renal disease.

### Pharmaceutical Compositions

Also provided herein are pharmaceutical compositions comprising a norepinephrine reuptake inhibitor (NRI) and a mineralocorticoid antagonist, and optionally a muscarinic receptor antagonist (MRA), as active ingredients. The active ingredients can be in a single composition or in separate compositions. In certain embodiments, the pharmaceutical compositions include atomoxetine or a pharmaceutically acceptable salt thereof and spironolactone or a pharmaceutically acceptable salt thereof, and optionally oxybutynin (e.g., (R)-oxybutynin) or a pharmaceutically acceptable salt thereof, as active ingredients.

Exemplary norepinephrine reuptake inhibitors (NRIs) include the selective NRIs, e.g., amedalin (UK-3540-1), atomoxetine (Strattera), CP-39,332, daledalin (UK-3557-15), edivoxetine (LY-2216684), esreboxetine, Jortalamine (LM-1404), nisoxetine (LY-94.939), reboxetine (Edronax, Vestra), talopram (Lu 3-010), talsupram (Lu 5-005), tandamine (AY-23,946), viloxazine (Vivalan); and the non-selective NRIs, e.g., amitriptiline, amoxapine, bupropion, ciclazindol, desipramine, desvenlafaxine, dexmethilphenidate, diethylpropion, doxepin, duloxetine, imipramine, levomilnacipran, manifaxine (GW-320,659), maprotiline, methylphenidate, milnacipran, nefazodone, nortriptyline, phendimetrazine, phenmetrazine, protryptyline, radafaxine (GW-353, 162), tapentadol (Nucynta), teniloxazine (Lucelan, Metatone) and venlafaxine; and pharmaceutically acceptable salts thereof.

In some embodiments, the NRI is atomoxetine or a pharmaceutically acceptable salt thereof.

Exemplary mineralocorticoid antagonists include spironolactone, eplerenone, canrenone, finerenone, mexrenone, canrenoic acid, drospirenone, prorenone, apararenone, and esaxerenone and pharmaceutically acceptable salts thereof.

In some embodiments, the mineralocorticoid antagonist is spironolactone or a pharmaceutically acceptable salt thereof.

Exemplary muscarinic receptor antagonists (MRAs) include atropine, propantheline, bethanechol, solifenacin, darifenacin, tolterodine, fesoterodine, trospium, and oxybutynin, and pharmaceutically acceptable salts thereof, which have activity on the M2 receptor. Other exemplary antimuscarinics include anisotropine, benztropine, biperiden, clidinium, cycrimine, dicyclomine, diphemanil, diphenidol, ethopropazine, glycopyrrolate, hexocyclium, isopropamide, mepenzolate, methixene, methscopolamine, oxyphencyclimine, oxyphenonium, procyclidine, scopolamine, tridihexethyl, and trihexyphenidyl, and pharmaceutically acceptable salts thereof.

In some embodiments, the muscarinic receptor antagonist is oxybutynin or (R)-oxybutynin, or a pharmaceutically acceptable salt thereof. As used herein, (R)-oxybutynin refers to the (R)-oxybutynin stereoisomer substantially free of other stereoisomers of oxybutynin. In some embodiments, the muscarinic receptor antagonist is fesoterodine.

In some embodiments, the pharmaceutical composition further comprises (iv) an additional active agent, which is a diuretic. The composition comprising a (iv) diuretic may be with or without an MRA. In some embodiments, the diuretic is selected from the group consisting of chlorothiazide, hydrochlorothiazide, bendroflumethiazide, chlorthalidone, methychlothiazide, polythiazide, triamterene, furosemide, ethacrynic acid, metolazone, bumetanide, indapamide, amiloride, and torsemide, or a pharmaceutically acceptable salt thereof.

Pharmaceutical compositions typically include a pharmaceutically acceptable carrier. As used herein the language “pharmaceutically acceptable carrier” includes saline, solvents, dispersion media, diluents, fillers, coatings, antibacterial and antifungal agents, isotonic and absorption delaying agents, and the like, compatible with pharmaceutical administration.

The active ingredients for use in the present invention may be provided as pharmaceutically acceptable salts. For example, in some embodiments, oxybutynin is oxybutynin chloride. In some embodiments, (R)-oxybutynin is (R)-oxybutynin chloride. In some embodiments, atomoxetine is atomoxetine hydrochloride.

Pharmaceutical compositions are typically formulated to be compatible with its intended route of administration. Examples of routes of administration include systemic oral or transdermal administration.

Methods of formulating suitable pharmaceutical compositions are known in the art, see, e.g., Remington: The Science and Practice of Pharmacy, 21st ed., 2005; and the books in the series Drugs and the Pharmaceutical Sciences: a Series of Textbooks and Monographs (Dekker, NY). For example, oral compositions generally include an inert diluent or an edible carrier. For the purpose of oral therapeutic administration, the active compound(s) can be incorporated with excipients and used in the form of pills, tablets, troches, or capsules, e.g., gelatin capsules. Oral compositions can also be prepared using a fluid carrier. In some embodiments, a composition according to the present invention may be a unit dosage form. In some embodiments, a composition according to the present invention may be a solid dosage form, e.g., a tablet or capsule.

Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

Systemic administration of the compounds as described herein can also be by transdermal means, e.g., using a patch, gel, or lotion, to be applied to the skin. For transdermal administration, penetrants appropriate to the permeation of the epidermal barrier can be used in the formulation. Such penetrants are generally known in the art. For example, for transdermal administration, the active compounds can formulated into ointments, salves, gels, or creams as generally known in the art. The gel and/or lotion can be provided in individual sachets, or via a metered-dose pump that is applied daily; see, e.g., Cohn et al., Ther Adv Urol. 2016 April; 8(2): 83-90.

In one embodiment, the therapeutic compounds are prepared with carriers that will protect the therapeutic compounds against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques, or obtained commercially, e.g., from Alza Corporation and Nova Pharmaceuticals, Inc. Liposomal suspensions can also be used as pharmaceutically acceptable carriers. These can be prepared according to methods known to those skilled in the art, for example, as described in U.S. Pat. No. 4,522,811.

The pharmaceutical compositions can be included in a container, pack, or dispenser together with instructions for administration or use in a method described herein.

In some embodiments, the pharmaceutical composition is for use in treating a condition associated with pharyngeal airway collapse. In some embodiments, the condition is sleep apnea (e.g., OSA) or snoring (e.g., simple snoring). In certain embodiments, provided herein is a pharmaceutical composition comprising atomoxetine or a pharmaceutically acceptable salt thereof and spironolactone or a pharmaceutically acceptable salt thereof, and optionally oxybutynin (e.g., (R)-oxybutynin) or a pharmaceutically acceptable salt thereof for use in treating sleep apnea (e.g., OSA) or snoring (e.g., simple snoring).

### Kits and Combinations

Also provided herein is a kit comprising (i) a norepinephrine reuptake inhibitor (NRI) and (ii) a mineralocorticoid antagonist, and optionally (iii) a muscarinic receptor antagonist. For example, the kit may comprise separate pharmaceutical compositions with each composition having a single active ingredient. The kits can be used for treating a subject having a condition associated with pharyngeal airway collapse. Various embodiments of kits will be apparent from the detailed description provided herein. In some embodiments, the kit further comprises an additional active agent, which is a diuretic (e.g., chlorothiazide, hydrochlorothiazide, bendroflumethiazide, chlorthalidone, methychlothiazide, polythiazide, triamterene, furosemide, ethacrynic acid, metolazone, bumetanide, indapamide, amiloride, or torsemide or a pharmaceutically acceptable salt thereof).

Also provided herein is a norepinephrine reuptake inhibitor (NRI) and a mineralocorticoid antagonist, and optionally a muscarinic receptor antagonist, for use in treating a subject having a condition associated with pharyngeal airway collapse. Further provided herein is a therapeutic combination of (i) a norepinephrine reuptake inhibitor (NRI) and (ii) a mineralocorticoid antagonist, and optionally (iii) a muscarinic receptor antagonist, for use in treating a subject having a condition associated with pharyngeal airway collapse. Various embodiments of combinations and therapeutic combinations will be apparent from the detailed description provided herein. In certain embodiments of the kits and combinations of the present invention, the NRI is atomoxetine or a pharmaceutically acceptable salt thereof, the mineralocorticoid antagonist is spironolactone or a pharmaceutically acceptable salt thereof, and the MRA, if present, is oxybutynin (e.g., (R)-oxybutynin) or a pharmaceutically acceptable salt thereof. In some embodiments, the combination or therapeutic combination further comprises an additional active agent, which is a diuretic (e.g., chlorothiazide, hydrochlorothiazide, bendroflumethiazide, chlorthalidone, methychlothiazide, polythiazide, triamterene, furosemide, ethacrynic acid, metolazone, bumetanide, indapamide, amiloride, or torsemide or a pharmaceutically acceptable salt thereof).

### EXAMPLES

The invention is further described in the following example, which does not limit the scope of the invention described in the claims.

### Example 1. Randomized Double-Blind 2-Period Multiple Dose Crossover Study to Evaluate the Efficacy and Safety of Atomoxetine+Spironolactone Vs. Atomoxetine in OSA Patients with Hypertension

The study of Example 1 is a randomized, double blind, placebo-controlled, 2-period, crossover study in patients with moderate to severe OSA. The study is designed to assess if the addition of spironolactone to atomoxetine can increase efficacy in a population with both hypertension and OSA. A screening visit and polysomnographic (PSG) exam will be conducted to establish that each participant meets study enrollment criteria. Each participant will then receive in random order for 10 nights the combinations consisting of the following:

A 3-day low-dose run-in period of atomoxetine 40 mg+spironolactone 25 mg followed by 7-day full dose period of atomoxetine 80 mg+spironolactone 50 mg.

A 3-day low-dose run-in period of atomoxetine 40 mg+spironolactone-matching placebo followed by 7-day full dose period of atomoxetine 80 mg+spironolactone-matching placebo.

A WatchPAT test and 24-hour BP monitoring will be will be conducted at home on the 9th day of treatment (6th day of full dose) of each treatment period. An inpatient PSG and WatchPAT will be performed on the 10th day of treatment (7th day of full dose) of each treatment period. There will be at least 3 weeks of washout period between the two treatment periods.

The following endpoints will be used.

| | Endpoints |
|---|---|
| Primary | Change in hypoxic burden (HB) 4% (scored in reference to |
| | AHI 4%), atomoxetine + spironolactone vs. atomoxetine |
| Secondary | Change in AHI 4% (hypopneas scored when associated with |
| | 4% O₂ desaturation), atomoxetine + spironolactone vs. |
| | atomoxetine |
| | Change in Oxygen Desaturation Index (ODI) |
| | Total time with SaO₂ <90%, PSG nights |
| | Proportion of participants with ≥50% reduction, AHI, |
| | hypoxic burden and ODI |
| Explor- | ESS |
| atory | Short SAQLI |
| | PGI-S |
| | PROMIS sleep impairment |
| | PROMIS sleep disturbance |
| | PROMIS fatigue |
| | AHI3 (hypopnea scored when associated with 3% O₂ |
| | desaturation) |
| | AHI3a (hypopnea scored when associated with 3% O₂ |
| | desaturation or arousal) |
| | OSA endotype endpoints (Vpassive, Vactive, Muscle |
| | Compensation, Loop Gain) |
| | PSG sleep and arousal parameters |
| | WatchPAT peripheral arterial tonometry signal |
| Safety | Physical exam, vital signs, clinical laboratory assessment |
| Endpoints | Spontaneous adverse events, including the post-dosing period |
| | PSG parameters: heart rate, ECG, EEG, oximetry |
| | Blood pressure |
| | Body weight |

Abbreviations: AHI = apnea-hypopnea index; ESS = Epworth Sleepiness Scale; SAQLI = Sleep Apnea Quality of Life Index; PGI-S = Patient Global Impression score ECG = electrocardiogram; EEG = electroencephalogram; ODI = Oxygen Desaturation Index; OSA = obstructive sleep apnea; PSG = polysomnography; SaO₂ = oxygen saturation.

Patients who meet all enrollment criteria will be randomized to receive in random order the following experimental treatments for 10 days, of which 9 will be administered at home and the last one will be administered in laboratory before a PSG night.

A 3-day low-dose run-in period of atomoxetine 40 mg+spironolactone 25 mg followed by 7-day full dose period of atomoxetine 80 mg+spironolactone 50 mg

A 3-day low-dose run-in period of atomoxetine 40 mg+spironolactone-matching placebo followed by 7-day full dose period of atomoxetine 80 mg+spironolactone-matching placebo

Study drug for the first dosing period is dispensed to patients at Visit 2 subsequent to randomization. Study drug consists of both supply for the 3-day run-in dose and the 7-day full-dose. WatchPAT and ambulatory blood pressure device are similarly dispensed to patients at Visit 2. Any unused study drug and the WatchPAT and blood pressure devices are returned at Visit 3. At Visit 3 study drug and devices for at-home use are similarly dispensed for the second dosing period, and at Visit 4 similarly returned to the study site.

Dosing of the study treatment will occur each night at the patient's usual bedtime, both during at-home nights and inpatient PSG nights. On the 9th day of dosing, at-home WatchPAT and 24-hour blood pressure monitoring will be conducted, with personnel from the study site contacting the patient by video call (or voice if video not feasible) to assist with and confirm proper use of the devices. The WatchPAT is removed by the patient in the morning, whereas the 24-hour blood pressure device remains in use until the patient arrives at the clinic for the PSG night (Visit 3 and Visit 4).

The evening of each inpatient PSG exam in the crossover period the ESS, PGI-S, SAQLI, and PROMIS instruments will be administered. Vital signs including seated blood pressure in triplicate, pulse, and respiratory rate, and weight are also measured the evening of each PSG exam. Each treatment period will be separated by at least a 3 week washout period.

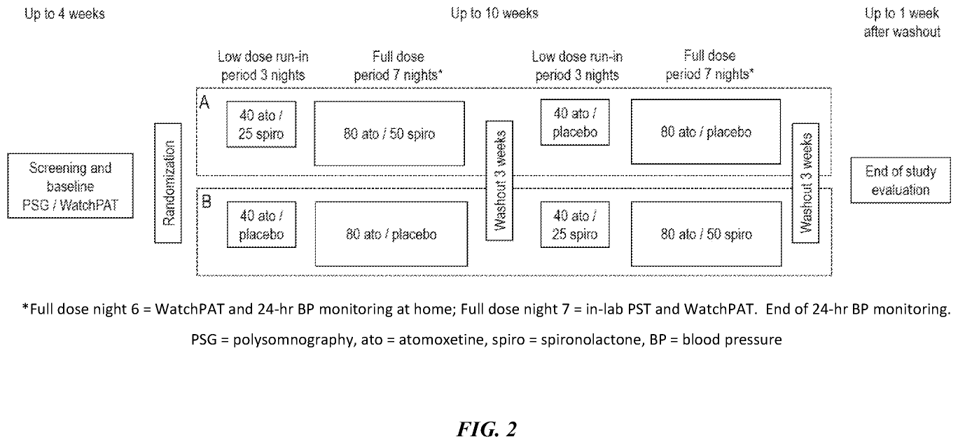

FIG. 2 shows an overview of the study design.

For the study treatment, a tablet of atomoxetine and spironolactone or spironolactone-matching placebo is taken immediately before the participant's planned bedtime. Spironolactone and placebo tablets, as well as, atomoxetine tablets will be over encapsulated for masking purpose.

| Study | Atomoxetine | | Spironolactone- |
|---|---|---|---|
| Treatment Name: | hydrochloride | Spironolactone | matching placebo |
| Dosage | Capsule | Capsule | Capsule |
| Formulation: | | | |
| Dosage Levels: | 40 and 80 mg | 25 and 50 mg | N/A |
| Route of | Oral | Oral | Oral |
| Administration: | | | |
| Dosing | 1 capsule | 1 capsule | 1 capsule |
| Instructions: | administered | administered | administered |
| | with up to 240 | with up to 240 | with up to 240 |
| | mL water | mL water | mL water |

Study procedures and timing are summarized in the following schedule of activities (SOA).

Schedule of Activities

| | | | 2-Way Crossover Period | | |
|---|---|---|---|---|---|
| | | | 3 day dosing | 7 day1 dosing period | |
| | Screening | | period (low | (full dose) followed | Wash- |
| Procedures | V1 | V2 | run-in dose) | by in-lab PSG V3 | out² |
| Trial Day (Visit Window) | Up to 4 weeks | | Up to 10 weeks | | |
| Informed consent | X | | | | |
| Demography | X | | | | |
| Physical exam | X | | | | |
| Medical history | X | | | | |
| Pregnancy test³ | X | | | | |
| Clinical laboratory testing | X | | | X | |
| 12 Lead ECG | X | | | | |
| Inpatient PSG Exam and | | X | | X | |
| WatchPAT⁴ | | | | | |
| Randomization | | X | | | |
| Study drug & device dispense/return | | X | | X | |
| HS study treatment⁵ | | | X | X | |
| At-home WatchPAT and 24-h BP⁶ | | | | X | |
| ESS, Short SAQLI, PGI-S, PROMIS | X | | | X | |
| sleep impairment, PROMIS sleep | | | | | |
| disturbance, PROMIS fatigue⁷ | | | | | |
| Vital signs8 and body weight | X | X | | X | |
| AE/SAE monitoring | | | X | X | X |
| Prior/concomitant medication | X | X | X | X | X |

| | 2-Way Crossover Period | | | |
|---|---|---|---|---|
| | | 7 day¹ dosing | | |
| | 3 day dosing | period (full dose) | | End of Study |
| | period (low | followed by in-lab | Wash- | Evaluation |
| Procedures | run-in dose) | PSG V4 | out² | Up to 1 week |
| Trial Day (Visit Window) | Up to 10 weeks | | | after washout |
| Informed consent | | | | |
| Demography | | | | |
| Physical exam | | | | |
| Medical history | | | | |
| Pregnancy test³ | | | | |
| Clinical laboratory testing | | X | | X |
| 12 Lead ECG | | | | |
| Inpatient PSG Exam and | | X | | |
| WatchPAT⁴ | | | | |
| Randomization | | | | |
| Study drug & device dispense/return | X | X | | |
| HS study treatment⁵ | X | X | | |
| At-home WatchPAT and 24-h BP⁶ | | X | | |
| ESS, Short SAQLI, PGI-S, PROMIS | | X | | |
| sleep impairment, PROMIS sleep | | | | |
| disturbance, PROMIS fatigue⁷ | | | | |
| Vital signs8 and body weight | | X | | X |
| AE/SAE monitoring | X | X | X | X |
| Prior/concomitant medication | X | X | X | X |

Abbreviations: AE = adverse event; BP = blood pressure; ECG = electrocardiogram; ESS = Epworth sleepiness scale; HS = Hora Somni; PGI-S = patient global impression of severity of OSA; PSG = polysomnography; SAE = serious adverse event; SAQLI = Sleep Apnea Quality of Life Index; WOCBP = women of childbearing potential; PROMIS = Patient Reported Outcomes Measurement Information System.
¹Up to 9 days if necessary for scheduling
²Each washout period is a minimum of 21 days
³WOCBP only
⁴PSG and WatchPAT used together each inpatient sleep study night
⁵Study medication administered immediately before lights out
⁶Both devices initiated at home on evening prior to in-lab PSG; site contacts patients by video call (or voice if video not feasible) at bedtime on night of use to confirm proper use
⁷Administer at similar time on evening of each crossover PSG, approximately 1 hour after admission
⁸Vital signs include the following: seated blood pressure in triplicate, pulse, respiratory rate; vital signs on PSG nights taken evening of admission to PSG lab

The following assessments will be made during the study.

Polysomnography: Standard overnight PSG recording and data interpretation will be performed in accordance with the American Academy of Sleep Medicine (AASM) scoring manual. Participants will be instrumented with standard PSG electrodes. Time of lights out will be established according to the participants' habitual schedule and kept constant across the PSG study nights. The participants will be given 8 hours of time-in bed. All PSG studies (including screening PSG) will be scored by on-site PSG technologists, blinded to treatment assignment. Scoring will be conducted according to the American Academy of Sleep Medicine manual scoring criteria.

WatchPAT: Standard WatchPAT recording will be performed at home and in-lab on PSG V2, V3 and V4, in accordance with AASM HSAT Clinical Practice Guidelines for Adults with OSA. Participants will be instrumented with standard WatchPAT device and sensors (finger probe and snoring/body position sensor). Participants will sleep at home as usual and in-lab also instrumented for full PSG. All WatchPAT studies will be scored by qualified on-site technologists, blinded to treatment assignment. Scoring will be conducted according to validated, evidence-based guidelines. WatchPAT is a sleep apnea test that utilizes the peripheral arterial signal (PAT). It measures up to 7 channels (PAT signal, heart rate, oximetry, actigraphy, body position, snoring, and chest motion) via 3 points of contact. WatchPAT provides AHI, AHIc, RDI, and ODI based upon true sleep time and sleep staging. WatchPAT is clinically validated with an 89% correlation to PSG. WatchPAT is a registered trademark of Itamar Medical Ltd.

Additional assessments will include safety, physical examinations, vital signs, electrocardiograms, clinical safety laboratory assessments, adverse events, and serious adverse events.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of treating a subject having a condition associated with pharyngeal airway collapse, the method comprising administering to a subject in need thereof an effective amount of (i) a norepinephrine reuptake inhibitor (NRI) and (ii) a mineralocorticoid antagonist.

2. The method of claim 1, wherein the NRI is a norepinephrine selective reuptake inhibitor (NSRI).

3. The method of claim 2, wherein the NSRI is selected from the group consisting of amedalin, atomoxetine, CP-39,332, daledalin, edivoxetine, esreboxetine, lortalamine, nisoxetine, reboxetine, talopram, talsupram, tandamine, and viloxazine, or a pharmaceutically acceptable salt thereof.

4. The method of claim 1, wherein the NRI is a norepinephrine non-selective reuptake inhibitor (NNRI) selected from the group consisting of amitriptiline, amoxapine, bupropion, ciclazindol, desipramine, desvenlafaxine, dexmethilphenidate, diethylpropion, doxepin, duloxetine, imipramine, levomilnacipran, manifaxine, maprotiline, methylphenidate, milnacipran, nefazodone, nortriptyline, phendimetrazine, phenmetrazine, protryptyline, radafaxine, tapentadol, teniloxazine, and venlafaxine, or a pharmaceutically acceptable salt thereof.

5. The method of claim 1, wherein the NRI is selected from the group consisting of atomoxetine or a pharmaceutically acceptable salt thereof and reboxetine or a pharmaceutically acceptable salt thereof.

6. The method of claim 5, wherein the NRI is atomoxetine or a pharmaceutically acceptable salt thereof.

7. The method of any one of claims 1-6, wherein the mineralocorticoid antagonist is selected from the group consisting of spironolactone, eplerenone, canrenone, finerenone, mexrenone, canrenoic acid, drospirenone, prorenone, apararenone, and esaxerenone, or a pharmaceutically acceptable salt thereof.

8. The method of claim 7, wherein the mineralocorticoid antagonist is spironolactone or a pharmaceutically acceptable salt thereof.

9. The method of claim 8, wherein the mineralocorticoid antagonist is spironolactone.

10. The method of any one of claims 1-9, further comprising administering to the subject (iii) a muscarinic receptor antagonist (MRA).

11. The method of claim 10, wherein the MRA is selected from the group consisting of atropine, propantheline, bethanechol, solifenacin, darifenacin, tolterodine, fesoterodine, trospium, and oxybutynin, or a pharmaceutically acceptable salt thereof.

12. The method of any claim 10, wherein the MRA is selected from the group consisting of anisotropine, benztropine, biperiden, clidinium, cycrimine, dicyclomine, diphemanil, diphenidol, ethopropazine, glycopyrrolate, hexocyclium, isopropamide, mepenzolate, methixene, methscopolamine, oxyphencyclimine, oxyphenonium, procyclidine, scopolamine, tridihexethyl, and trihexyphenidyl, or a pharmaceutically acceptable salt thereof.

13. The method of claim 11, wherein the MRA is oxybutynin or a pharmaceutically acceptable salt thereof.

14. The method of claim 13, wherein the MRA is (R)-oxybutynin or a pharmaceutically acceptable salt thereof.

15. The method of any one of claims 1-14, further comprising administering to the subject an additional active agent, which is a diuretic.

16. The method of claim 15, wherein the diuretic is selected from the group consisting of chlorothiazide, hydrochlorothiazide, bendroflumethiazide, chlorthalidone, methychlothiazide, polythiazide, triamterene, furosemide, ethacrynic acid, metolazone, bumetanide, indapamide, amiloride, and torsemide or a pharmaceutically acceptable salt thereof.

17. The method of any one of claims 1-16, wherein the atomoxetine or pharmaceutically acceptable salt thereof is administered at a dose of from about 20 to about 200 mg.

18. The method of claim 17 wherein the atomoxetine or pharmaceutically acceptable salt thereof is administered at a dose of from about 25 to about 100 mg.

19. The method of any one of claims 1-18, wherein the spironolactone or pharmaceutically acceptable salt thereof is administered at a dose of from about 10 to about 100 mg.

20. The method of claim 19, wherein the spironolactone or pharmaceutically acceptable salt thereof is administered at a dose of from about 20 to about 80 mg.

21. The method of any one of claims 10-20, wherein the oxybutynin or pharmaceutically acceptable salt thereof is administered at a dose of from about 1 to about 15 mg.

22. The method of claim 21, wherein the oxybutynin or pharmaceutically acceptable salt thereof is administered at a dose of from about 2 mg to about 10 mg.

23. The method of any one of claims 10-20, wherein the (R)-oxybutynin or pharmaceutically acceptable salt thereof is administered at a dose of from about 0.5 to about 10 mg.

24. The method of claim 23, wherein the (R)-oxybutynin or pharmaceutically acceptable salt thereof is administered at a dose of from about 1 mg to about 5 mg.

25. The method of any one of claims 1-24, wherein the NRI and mineralocorticoid antagonist are administered in a single composition.

26. The method of any one of claims 10-25, wherein the NRI, MRA, and mineralocorticoid antagonist are administered in a single composition.

27. The method of claim 25 or 26, wherein the single composition is an oral administration form.

28. The method of claim 27, wherein the oral administration form is a syrup, pill, tablet, troche, capsule, or patch.

29. The method of any one of claims 1-28, wherein the condition associated with pharyngeal airway collapse is sleep apnea.

30. The method of claim 29, wherein the condition associated with pharyngeal airway collapse is obstructive sleep apnea (OSA).

31. The method of any one of claims 1-28, wherein the condition associated with pharyngeal airway collapse is snoring.

32. The method of claim 31 wherein the condition associated with pharyngeal airway collapse is simple snoring.

33. The method of any one of claims 1-32, wherein the subject is in a non-fully conscious state, such as sleep.

34. The method of any one of claims 1-33, wherein the subject has hypertension.

35. A pharmaceutical composition comprising (i) a norepinephrine reuptake inhibitor (NRI) and (ii) a mineralocorticoid antagonist, in a pharmaceutically acceptable carrier.

36. The composition of claim 35, wherein the NRI is a norepinephrine selective reuptake inhibitor (NSRI).

37. The composition of claim 36, wherein the NSRI is selected from the group consisting of amedalin, atomoxetine, CP-39,332, daledalin, edivoxetine, esreboxetine, lortalamine, nisoxetine, reboxetine, talopram, talsupram, tandamine, and viloxazine, or a pharmaceutically acceptable salt thereof.

38. The composition of claim 35, wherein the NRI is a norepinephrine non-selective reuptake inhibitor (NNRI) selected from the group consisting of amitriptiline, amoxapine, bupropion, ciclazindol, desipramine, desvenlafaxine, dexmethilphenidate, diethylpropion, doxepin, duloxetine, imipramine, levomilnacipran, manifaxine, maprotiline, methylphenidate, milnacipran, nefazodone, nortriptyline, phendimetrazine, phenmetrazine, protryptyline, radafaxine, tapentadol, teniloxazine, and venlafaxine, or a pharmaceutically acceptable salt thereof.

39. The composition of claim 35, wherein the NRI is selected from the group consisting of atomoxetine or a pharmaceutically acceptable salt thereof and reboxetine or a pharmaceutically acceptable salt thereof.

40. The composition of claim 39, wherein the NRI is atomoxetine or a pharmaceutically acceptable salt thereof.

41. The composition of any one of claims 35-40, wherein the mineralocorticoid antagonist is selected from the group consisting of spironolactone, eplerenone, canrenone, finerenone, mexrenone, canrenoic acid, drospirenone, prorenone, apararenone, and esaxerenone, or a pharmaceutically acceptable salt thereof.

42. The composition of claim 41, wherein the mineralocorticoid antagonist is spironolactone or a pharmaceutically acceptable salt thereof.

43. The composition of claim 42, wherein the mineralocorticoid antagonist is spironolactone.

44. The composition of any one of claims 35-43, further comprising (iii) a muscarinic receptor antagonist (MRA).

45. The composition of claim 44, wherein the MRA is selected from the group consisting of atropine, propantheline, bethanechol, solifenacin, darifenacin, tolterodine, fesoterodine, trospium, and oxybutynin, or a pharmaceutically acceptable salt thereof.

46. The composition of any claim 44, wherein the MRA is selected from the group consisting of anisotropine, benztropine, biperiden, clidinium, cycrimine, dicyclomine, diphemanil, diphenidol, ethopropazine, glycopyrrolate, hexocyclium, isopropamide, mepenzolate, methixene, methscopolamine, oxyphencyclimine, oxyphenonium, procyclidine, scopolamine, tridihexethyl, and trihexyphenidyl, or a pharmaceutically acceptable salt thereof.

47. The composition of claim 45, wherein the MRA is oxybutynin or a pharmaceutically acceptable salt thereof.

48. The composition of claim 47, wherein the MRA is (R)-oxybutynin or a pharmaceutically acceptable salt thereof.

49. The composition of any one of claims 35-48, further comprising an additional active agent, which is a diuretic.

50. The composition of claim 49, wherein the diuretic is selected from the group consisting of chlorothiazide, hydrochlorothiazide, bendroflumethiazide, chlorthalidone, methychlothiazide, polythiazide, triamterene, furosemide, ethacrynic acid, metolazone, bumetanide, indapamide, amiloride, and torsemide or a pharmaceutically acceptable salt thereof.

51. The composition of any one of claims 35-50, wherein the atomoxetine or pharmaceutically acceptable salt thereof is present in an amount of from about 20 to about 200 mg.

52. The composition of claim 51, wherein the atomoxetine or pharmaceutically acceptable salt thereof is present in an amount of from about 25 to about 100 mg.

53. The composition of any one of claims 35-52, wherein the spironolactone or pharmaceutically acceptable salt thereof is present in an amount of from about 10 to about 100 mg.

54. The composition of claim 53, wherein the spironolactone or pharmaceutically acceptable salt thereof is present in an amount of from about 20 to about 80 mg.

55. The composition of any one of claims 44-54, wherein the oxybutynin or pharmaceutically acceptable salt thereof is present in an amount of from about 1 to about 15 mg.

56. The composition of claim 55, wherein the oxybutynin or pharmaceutically acceptable salt thereof is present in an amount of from about 2 mg to about 10 mg.

57. The composition of any one of claims 44-54, wherein the (R)-oxybutynin or pharmaceutically acceptable salt thereof is present in an amount of from about 0.5 to about 10 mg.

58. The composition of claim 57, wherein the (R)-oxybutynin or pharmaceutically acceptable salt thereof is present in an amount of from about 1 mg to about 5 mg.

59. The composition of any one of claims 35-58, wherein the NRI and mineralocorticoid antagonist are formulated in a single composition.

60. The composition of any one of claims 44-59, wherein the NRI, MRA, and mineralocorticoid antagonist are formulated in a single composition.

61. The composition of claim 59 or 60, wherein the single composition is an oral administration form.

62. The composition of claim 61, wherein the oral administration form is a syrup, pill, tablet, troche, capsule, or patch.

63. The composition of any one of claims 35-62, for use in treating a subject having a condition associated with pharyngeal airway collapse.

64. The composition for use of claim 63, wherein the condition associated with pharyngeal airway collapse is sleep apnea.

65. The composition for use of claim 64, wherein the condition associated with pharyngeal airway collapse is obstructive sleep apnea (OSA).

66. The composition for use of claim 63, wherein the condition associated with pharyngeal airway collapse is snoring.

67. The composition for use of claim 66, wherein the condition associated with pharyngeal airway collapse is simple snoring.

68. The composition for use of any one of claims 63-67, wherein the subject is in a non-fully conscious state, such as sleep.

69. The composition for use of any one of claims 63-68, wherein the subject has hypertension.

70. A kit comprising (i) a norepinephrine reuptake inhibitor (NRI) and (ii) a mineralocorticoid antagonist, and optionally (iii) a muscarinic receptor antagonist (MRA).

71. The kit of claim 70, for use in treating a subject having a condition associated with pharyngeal airway collapse.

72. A norepinephrine reuptake inhibitor (NRI) and a mineralocorticoid antagonist, and optionally a muscarinic receptor antagonist (MRA), for use in treating a subject having a condition associated with pharyngeal airway collapse.

73. A therapeutic combination of (i) a norepinephrine reuptake inhibitor (NRI) and (ii) a mineralocorticoid antagonist, and optionally (iii) a muscarinic receptor antagonist (MRA), for use in treating a subject having a condition associated with pharyngeal airway collapse.
